(19) United States
(12) Patent Application Publication
(10) Pub. No.: US 2005/0059725 A1
(43) Pub. Date: Mar. 17, 2005

(54) Methods of using and preparing thiolutin dioxide

(75) Inventors: Claudia Eder, Hofheim (DE); Michael Kurz, Hofheim (DE); Joachim Wink, Rodermark (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/973,309

(22) Filed: Oct. 26, 2004

Related U.S. Application Data
(63) Continuation of application No. 10/092,882, filed on Mar. 8, 2002.

(30) Foreign Application Priority Data
Mar. 9, 2001 (EP) 01105959.9

(51) Int. Cl. A61K 31/407

(52) U.S. Cl. 514/412

(57) ABSTRACT

The present invention relates to the use of thiolutin dioxide and its derivatives in the manufacture of a medicament. Such medicaments are useful, for example, in the treatment of CNS disorders. The present invention also relates to a process for the production of thiolutin dioxide and its derivatives by fermentation of microorganisms, such as the microorganism Nocardiopsis species ST 100692 (DSM 13834).

This application is a continuation of U.S. patent application No. 10/092,882, filed Mar. 8, 2002, which claims the benefit of priority from European Patent Application No. 01105959.9, filed Mar. 9, 2001.

The present invention relates to the use of thiolutin dioxide and its derivatives in the manufacture of a medicament for the treatment of CNS disorders; to a process for preparing thiolutin and its derivatives by fermentation of microorganisms; and to the microorganism Nocardiopsis species ST 100692 (DSM 13834).

Thiolutin is a natural compound which is available commercially (Apin Chemicals, UK; CMS Speciality Chemicals, UK; Ubichem plc, UK). Thiolutin dioxide is also a known compound. One known process for the preparation of thiolutin dioxide is by oxidation of thiolutin with m-chlorperbenzoic acid (Yield 30%) as described in Schachtner et al. (1999) J. Heterocycl. Chem., pp. 161-175.

Thiolutin dioxide has previously been described as having medicinal properties. For instance, thiolutin dioxide has been described as an antineoplastic agent (WO 99/12543) and an antibacterial and antifungal agent (WO 96/32396).

It has now surprisingly been found that thiolutin dioxide is an effective inhibitor of neurolysin. Neurolysin belongs to the family of zinc-containing metalloproteases. It plays a likely role in the physiological inactivation of neurotensin, an endogenous antipsychotic agent. Thiolutin dioxide inhibits neurolysin-mediated inactivation of neurotensin. Thiolutin dioxide is therefore useful in the treatment of neurodegenerative diseases such as Parkinson's and Alzheimer's. Thiolutin dioxide is also selective in that it does not block other zinc-containing metalloproteases such as enkephalinase or angiotensin converting enzymes.

It has now also been found that the microorgansim Nocardiopsis species ST 100692 (DSM 13834) is able to produce relatively high yields of thiolutin dioxide.

The present invention accordingly relates to the use of a compound of formula I 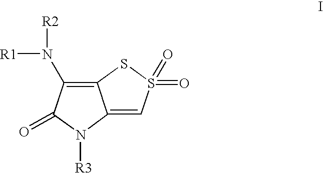
wherein R1, R2 and R3 are independently selected from: H, alkyl, and acyl; and physiologically tolerated salts thereof. The compound is a medicament and may be used, for example, to treat CNS disorders.

In one embodiment, compounds of formula I include those in which R1 is acyl, R2 is H and/or R3 is alkyl; and the physiologically tolerated salts thereof.

The acyl radicals in the compounds of formula I may have 2 to 10 carbon atoms, optionally 2 to 6 carbon atoms, and can be straight-chain, branched, saturated, or unsaturated at one or two positions.

An acyl radical with 2 carbon atoms includes, for example, an acetyl radical.

Examples of saturated, unbranched acyl radicals include, for example, an acetic acid residue, propionic acid residue, butyric acid residue, valeric acid residue, caproic acid residue, enanthic acid residue, caprylic acid residue, pelargonic acid residue and capric acid.

Examples of unbranched acyl residues which are unsaturated at one position include an acrylic acid residue and crotonic acid residue.

An example of an unbranched acyl radical which is unsaturated in two positions is a sorbic acid residue.

The alkyl radicals in the compounds of formula I may have from 1 to 6 carbon atoms and can be straight-chain or branched. Further, the alkyl radicals include saturated as well as unsaturated groups, which latter groups contain one or two double bonds. Examples of alkyl radicals containing from 1 to 6 carbon atoms include methyl, ethyl, propyl, butyl, pentyl and hexyl, the n-isomers of all these radicals, isopropyl, isobutyl, 1-methylbutyl, isopentyl, neopentyl, 2,2-dimethylbutyl, 2-methylpentyl, 3-methylpentyl and isohexyl.

Unsaturated alkyl radicals include, for example, alkenyl residues such as vinyl, 1-propenyl, 2-propenyl (=allyl), 2-butenyl, 3-butenyl, 2-methyl-2-butenyl, 3-methyl-2-butenyl, 5-hexenyl or 1,3-pentadienyl.

In one embodiment, the invention relates to the use of a compound of formula II 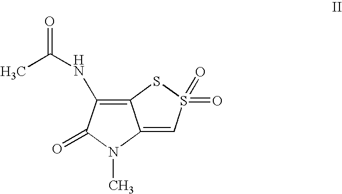
or a physiologically tolerated salt thereof, in the manufacture of a medicament. The medicament may be used to treat CNS disorders.

The compounds of formula I are useful in treating disorders characterized by higher than normal levels of circulating neurolysin. CNS disorders which may be treated with the compounds of formula I include psychotic disorders such as schizophrenia, and neurodegenerative disorders such as Alzheimer's and Parkinson's disease.

The compounds of formula I are obtainable by fermentation of a microorganism, such as Nocardiopsis species ST 100692 (DSM 13834), or one of its variants or mutants under suitable conditions. The compounds may be isolated and converted, where appropriate, into a physiologically tolerated salt of a compound of formula I, a derivative, or a physiologically tolerated salt of the derivative.

The microorganism Nocardiopsis species ST 100692 was deposited on Nov. 13, 2000, under the conditions of the Budapest treaty, at the Deutsche Sammlung von Mikroorganismen und Zellkulturen, Mascheroder Weg 1b, D-38124 Braunschweig, under the number DSM 13834.

A taxonomic examination of the microorganism Nocardiopsis species ST 100692 (DSM 13834) by analysis of fatty acids using gas chromatography showed the characteristic acids to be: 14:0 iso, 15:0 anteiso, 15:0 iso, 16:0, 16:0 iso, 17:0, 17:0 iso, 17:0 anteiso and 18:0. The term iso refers to a fatty acid hydrocarbon chain containing a methyl group substituted at the second to the last carbon in the chain. Thus, “17:0 iso” refers to a fatty acid hydrocarbon chain containing 17 carbons and having a methyl group substituted on the 15^{th} (2^{nd} to last) carbon. The term “anteiso” refers to a fatty acid hydrocarbon chain containing a methyl group substituted at the third to the last carbon in the chain. The colony colour is chrome yellow forming white aerial mycelium especially on ISP 2 (yeast-malt) and ISP 3 (oatmeal) medium.

The invention accordingly relates to a process for producing a compound of formula I. The process comprises cultivating a microorganism, such as Nocardiopsis species ST 100692 (DSM 13834), or a mutant or variant thereof, in an aqueous nutrient medium; isolating and purifying at least one target compound from the microorganism or nutrient medium; and optionally converting the compound into a physiologically tolerated salt of a compound of formula I, derivative or physiologically tolerated salt of the derivative.

In addition to producing thiolutin dioxide, Nocardiopsis species ST 100692 (DSM 13834) also produces thiolutin under the conditions of fermentation described. The thiolutin obtained may be isolated and converted into thiolutin dioxide by methods known to the skilled artisan.

An alternative process for producing a compound of formula I according to the present invention comprises cultivating a microorganism, such as Nocardiopsis species ST 100692 (DSM 13834), or a mutant or variant thereof, in an aqueous nutrient medium; isolating and purifying thiolutin from the microorganism; converting thiolutin into at least one target compound; and optionally further converting the target compound into a physiologically tolerated salt, derivative, or physiologically tolerated salt of the derivative.

In place of the strain DSM 13834, it is also possible to employ its mutants and variants that synthesize the compounds according to the invention. Such mutants can be generated by methods known in the art. For example, mutants may be generated by physical means such as irradiation; and by chemical means such as ethyl methanesulfonate (EMS), 2-hydroxy-4-methoxybenzophenone (MOB), or N-methyl-N′-nitro-N-nitroguanidine (MNNG).

Screening for mutants and variants which produce the compounds according to the invention can be accomplished by testing the biological activity of the active substances which have accumulated in the culture broth. For example, the compound in the broth may be tested for inhibition of neurolysin by the method described below.

The microorganisms are typically fermented in an appropriate nutrient medium. Suitable sources of carbon for aerobic fermentation include assimilable carbohydrates and sugar alcohols such as glucose, lactose or D-mannitol; and carbohydrate-containing natural products such as malt extract. Suitable nitrogen-containing nutrients include amino acids; peptides, proteins, and their degradation products such as peptones or tryptones; meat extracts; ground seeds, such as those from corn, wheat, soybean or cotton plants; distillation residues from the production of alcohol, meat meals, or yeast extracts; and also ammonium salts and nitrates. Inorganic salts which the nutrient solution may contain include, for example, chlorides, carbonates, sulphates or phosphates of the alkali metals or alkaline earth metals, iron, zinc, cobalt and manganese.

The formation of thiolutin dioxide is achieved, for example, in a nutrient medium which contains about 0.5 to 5% glucose, optionally 1 to 2%; 0.5 to 5% soybean meal, optionally 1 to 2%; 0.1 to 1.5% corn steep (fluid), optionally 0.3% to 0.8%; 0.05 to 1.0% calcium carbonate, optionally 0.1 to 0.5%; and 0.05 to 1% sodium chloride, optionally 0.3% to 0.8%; in each case based on the weight of the complete nutrient solution.

The cultivation takes place aerobically, for example, submerged with shaking or stirring in shaken flasks or fermenters, with introduction of air or oxygen as appropriate. The fermentation can be carried out, for example, in wide-necked bottles or round-bottomed flasks of various volumes, in glass fermenters, or stainless steel tanks. It can be carried out in a temperature range of about 20 to 35° C., optionally at about 25 to 30° C. The pH should be between 4 and 10, optionally between 6 and 8. The microorganisms are typically cultivated under these conditions for a period of from 20 to 200 hours, optionally 24 to 150 hours.

Cultivation is typically carried out in several stages. For example, a sporulated mycelium can be obtained by allowing the strain to grow for about 1 to 40 days (e.g., 5 to 12 days) on a solid or liquid nutrient medium such as yeast-malt agar or potato-dextrose agar. A preculture is then obtained, for example, by transferring the sporulated mycelium into a nutrient solution and allowing it to grow for about 20 to 120 hours, or about 24 to 90 hours. One or more precultures in the liquid nutrient medium are then diluted into the actual production medium (the main culture) at a particular ratio, such as 1:10 by volume.

The progress of the fermentation and the formation of the compounds according to the invention can be followed according to methods known to the skilled artisan, such as, for example, by testing the biological activity in bioassays, by chromatographic methods such as thin-layer chromatography (TLC), or by high performance liquid chromatography (HPLC).

The compounds of formula I may occur both in the mycelium and in the culture filtrate, but the larger amounts are usually found in the biomass (mycelium). It is therefore typical to separate the latter from the former by filtration or centrifugation. The filtrate is extracted with a water-immiscible solvent such as 1-butanol, ethyl acetate, chloroform or the like. The mycelium is typically extracted with methanol or acetone, but it is also possible to use the above mentioned water-immiscible solvents.

The extractions can be carried out in a wide pH range, but it is typical to operate in a neutral medium, such as between pH 4 and pH 8. Also, the organic extracts can, for example, be concentrated in vacuum and dried.

One purification method involves chromatography on adsorption resins such as on Diaion® HP-20 (Mitsubishi Casei., Tokyo), Amberlite® XAD7 (Rohm and Haas, USA), and Amberchrom® CG, (Toso Haas, Philadelphia, USA). Also suitable are numerous reverse phase supports, for example RP18, and others that have become generally used, for example, in high pressure liquid chromatography (HPLC).

The compounds of the formula I can be isolated and purified according to the methods described above or by other methods known to the skilled artisan.

Derivatives of thiolutin dioxide are also encompassed by formula I, above. These derivatives have the same effect, or are converted under mild conditions into compounds having the same effect, as thiolutin dioxide. The derivatives may be prepared by processes well known to the skilled artisan. Examples of preparative processes of some of the thiolutin dioxide derivatives covered by formula I are given below:

1) The (exocyclic) acetyl group of thiolutin dioxide can be cleaved with an acid or base as described, e.g., in Protective Groups in Organic Synthesis (1999) 3rd Edition, T. Greene & P. Wuts, John Wiley & Sons, pp 553-555.

2) The free amino group of thiolutin dioxide can be alkylated, for example, via reductive alkylation as described, e.g., in Advanced Organic Chemistry (1992) 4th Edition, J. March, John Wiley & Sons, pp 898-900.

3) The amino group of thiolutin dioxide can be acylated, e.g., with acid chlorides or anhydrides by standard procedures well known to one skilled in the art.

Further derivatives of compounds of formula I include those compounds which result from a reduction of at least one double bond in a compound of formula I, such as thiolutin dioxide, by methods given in the literature. For example, suitable reduction reactions are described by P. N. Rylander (Hydrogenation Methods (1985), Academic Press, New York, Chap. 2). The derivatives may also be prepared by dehydrohalogenation, using methods such as those described by H. O. House, (Modern Synthetic Reactions (1972), W.A. Benjymin, Inc., New York, pp 446-452).

The compounds according to the present invention may be converted into pharmaceutically acceptable salts. The salts can be prepared by standard procedures known to one skilled in the art.

Physiologically tolerated salts of the compounds of the formula I include both the organic and the inorganic salts thereof as described, for example, in Remington's Pharmaceutical Sciences (17th edition, page 1418 (1985)). Sodium and potassium salts, for example, may be prepared by treating the compounds according to the invention with suitable sodium or potassium bases.

A further aspect of the present invention is the use of a prodrug of a compound of formula I. Such a prodrug can be metabolized in vivo to a compound of formula I, such as thiolutin dioxide. These prodrugs may themselves be active or inactive.

The compounds of formula I may exist in various polymorphous forms, for example as amorphous and crystalline polymorphous forms. All polymorphous forms of the compounds of formula I fall within the scope of the invention and are a further aspect of the invention.

The compounds according to the present invention and their pharmaceutically acceptable salts and derivatives can be administered to animals, for example, to mammals, and in particular to humans. The compounds may be administered as pharmaceuticals either individually, in mixtures with other compounds of formula I, or in mixtures with other pharmaceutically active compounds.

Suitable pharmaceutical compositions accordingly comprise an effective amount of one or more of the compounds of formula I or II, or pharmaceutically acceptable salts thereof, together with a pharmaceutically acceptable carrier.

The compounds according to the invention can be administered orally, intramuscularly, intravenously or by other modes of administration. Pharmaceutical compositions which contain these compounds, or pharmaceutically acceptable salts or derivatives thereof, optionally with other pharmaceutically active substances, can be prepared by mixing the active compounds with one or more pharmacologically tolerated auxiliaries and/or excipients. The mixture can then be converted into suitable pharmaceutical forms such as tablets, coated tablets, capsules, granules, powders, emulsions, suspensions or solutions.

Examples of auxiliaries and/or excipients which may be used include fillers, emulsifiers, lubricants, masking flavours, colorants and buffer substances, tragacanth, lactose, talc, agar-agar, polyglycols, ethanol and water. Suitable dosage forms for parenteral administration include suspensions or solutions in water. It is also possible to administer the active substances in a suitable form without vehicles or diluents, for example, in capsules.

A method for producing suitable pharmaceutical dosage forms comprises mixing at least one of the compounds according to the present invention with a pharmaceutically suitable and physiologically tolerated carrier and, where appropriate, additional active substances, additives or excipients.

As is customary, the pharmaceutical composition, the method of its administration, and the suitable dosage range depend on the species to be treated and the state of the respective condition or disease. Such parameters can be optimized using methods known in the art. Using solid dosage forms, e.g., tablets or capsules, up to 500 mg, optionally 0.1 to 250 mg, can be administered per day. For parenteral application up to 300 mg, optionally 0.5 to 150 mg, can be given per day.

The following are illustrative examples of the present invention, but are not intended to limit the scope thereof:

### Example 1

### Production of a Spore Suspension of Nocardiopsis

A 100 ml of nutrient solution (4 g/l yeast extract, 15 g/l soluble starch, 1 g/l K₂HPO₄, 0.5 g/l MgSO₄×7 H₂O in 1000 ml water, pH before sterilization 7.0) in a 300 ml sterile Erlenmeyer flask were innoculated with the strain Nocardiopsis species DSM 13834 and incubated at 28° C. and 180 rpm on a rotary shaker for 5 days. Subsequently, 1.5 ml of this culture was diluted with 1.5 ml of 99% glycerin and stored at −20° C.

### Example 2

### Production of a Culture or Pre-culture of Nocardiopsis

A sterile Erlenmeyer flask containing 100 ml of the following nutrient solution: 15 g/l soya flour, 15 g/l glucose, 5 g/l corn steep fluid, 5 g/l NaCl and 2 g/l CaCO₃, was innoculated with a culture which had grown in a slant tube (same nutrient solution but with 2% agar) or in 1 ml of a glycerin culture (see Example 1) and incubated on a shaker at 180 rpm and 25° C. The maximum production of thiolutin dioxide was reached after about 96 hours. A 72-hour old submerged culture (produced according to the process described for the shake culture but with the following medium: 15 g/l glucose, 15 g/l soya flour, 5 g/l corn steep, 2 g/l CaCO₃ and 5 g/l NaCl, pH 7.5) was sufficient for inoculating 10 and 100 liter fermenters with an inoculum of about 5%.

### Example 3

### Production of Thiolutin Dioxide

A 200 liter fermenter was operated under the following conditions:

| | | |
|---|---|---|
| Nutrient medium: | Soya flour | 15 g/l |
| | Glucose | 15 g/l |
| | Corn steep | 5 g/l |
| | NaCl | 5 g/l |
| | CaCO₃ | 2 g/l |

| | |
|---|---|
| | pH 7.2 (before sterilization) |
| Incubation time: | 60-80 hours |
| Incubation temperature: | 28° C. |
| Stirrer speed: | 50 rpm |
| Aeration: | 150 l/min |

Foaming was suppressed by a repeated addition of a few drops of 1 to 2 ml of ethanolic polyol solution. The production maximum was reached after 69 hours.

### Example 4

### Isolation of Thiolutin Dioxide

A 3 liter culture solution obtained in Example 3 was lyophilised. The lyophilisate was subsequently extracted with methanol (2-3l). The methanol extract was reduced in a vacuum and diluted with water containing a methanol content of 10%. The diluted extract was loaded onto a 1 liter column packed with the adsorption resin CHP-20P. Elution was accomplished by applying a solvent gradient from water to acetonitrile. Column flow through (30 ml/min) was collected in fractions of 30 ml. The desired compond-containing fractions were collected, concentrated in vacuo and lyophilised to give approximately 30 mg of yellow-brown powder. The resultant powder was loaded onto a column packed with LUNA® 10 uC18(2) (width×height=21 mm×250 mm) and eluted with a gradient of from 10 to 60% acetonitrile in 0.1% ammonium acetate/ water. The flow rate of the elution medium was 25 ml/min and flow through from the column was collected in fractions of 25 ml. Thiolutin dioxide was found in fractions 15 and 16. Lyophilisation of these fractions gave 1.8 mg >95% pure thiolutin dioxide.

The physicochemical and spectroscopic properties of thiolutin dioxide can be summarised as follows:
Molecular formula: C₈H₈N₂O₄S₂
Molecular weight: 260.3
UV-Maxima: 230, 302, 388 nm

1H- and 13-C NMR: see Table 1

TABLE 1
1H- and 13-CNMR: Chemical shifts of
thiolutin dioxide in DMSO at 300K

| | 1H | 13C |
|---|---|---|
| 1 | 2.10 | 22.51 |
| 2 | — | 170.46 |
| 3 | 10.65 | — |
| 4 | — | 123.01 |
| 5 | — | 164.27 |
| 6 | 3.10 | 27.85 |
| 7 | — | 145.48 |
| 8 | — | 114.05 |
| 9 | 7.55 | 109.58 |

### Example 5

### Thiolutin Dioxide As A Neurolysin Inhibitor.

An assay was performed on a CyBio pipetting system in a 384-well plate format in a final assay volume of 16 μl. In brief, 4 μl of appropriately diluted microbial extracts (dilution in 50 mM Tris buffer, pH 7.5) were distributed in wells of test plates (Greiner, white 384-small volume well plates). Thereafter, 4 μl of neurolysin (pre-diluted 1:5, 360 ng of protein) were added to each well. After a 10 minute pre-incubation of the samples and the enzyme at room temperature, the reaction was initiated by the addition of 8 μl of substrate (Mcc-Pro-Leu-Gly-D-Lys (Dnp)—OH, CALBIOCHEM) in Tris buffer. The final test concentration of the substrate was 4 μM.

After pipetting, the plates were immediately placed in a fluorometer (SpectraFluorplus, SLT) and the initial amount of fluorescence was read (λₑₓ: 360 nm/λₑₘ405 nm). The reaction was then allowed to proceed at 30° C. for 30 min, and a final fluorescence reading was taken.

The data are first blank corrected. Then, after subtracting the values at the zero time point from the respective values after 30 min, sample inhibition activity was expressed as
100−(Net Intensity compound/Net Intensity control)×100(%)

Each test plate contained a reasonable number of positive controls and blanks (buffer instead of enzyme).

The IC₅₀ of thiolutin dioxide was found to be 0.6 μM.

1. A method of treating a CNS disorder selected from the group consisting of schizophrenia and Parkinson's disease comprising administering, to a patient in need of such a treatment, a compound of formula I, or any physiologically tolerated salts thereof, 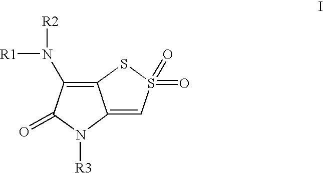
wherein
R1, R2 and R3 are independently selected from H, alkyl, and acyl.

2. The method of claim 1, wherein R1 is acyl, R2 is H, and R3 is alkyl in the compound of formula I.

3. The method of claim 1, wherein the compound is thiolutin dioxide of formula II 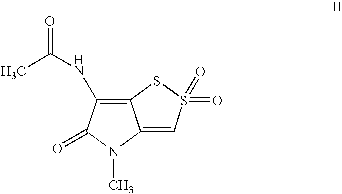
or any physiologically tolerated salts thereof.
